(19) United States
(12) Patent Application Publication
(10) Pub. No.: US 2023/0157675 A1
(43) Pub. Date: May 25, 2023

(54) SYSTEM AND METHOD TO RETRIEVE MEDICAL X-RAYS

(71) Applicant: GSI Technology Inc., Sunnyvale, CA (US)

(72) Inventors: Elona EREZ, Tel Aviv (IL); Avidan AKERIB, Tel Aviv (IL)

(21) Appl. No.: 17/902,929

(22) Filed: Sep. 5, 2022

Related U.S. Application Data
(60) Provisional application No. 63/246,854, filed on Sep. 22, 2021.
(60) Provisional application No. 63/403,763, filed on Sep. 4, 2022.

(51) Int. Cl. A61B 8/00; A61B 5/00

(52) U.S. Cl. CPC A61B 8/565; A61B 5/7267; G16H 30/20

(57) ABSTRACT

A system to retrieve medical X-rays includes a trained convolutional neural network (CNN), a balancing feature generator, a balancing type selector, and a K-Nearest Neighbor (KNN) classifier. The trained CNN encodes a plurality of diagnosed X-ray images into a plurality of candidate embeddings, and encodes a partially diagnosed X-ray image into a query embedding. The balancing feature generator produces a plurality of virtual candidate embeddings from the query embedding and the plurality of candidate embeddings. The balancing type selector selects a subset of the plurality of virtual candidate embeddings. The KNN classifier performs a KNN search between the query embedding and a plurality of the candidate embeddings and the subset of the plurality of virtual candidate embeddings.

### CROSS REFERENCE TO RELATED APPLICATIONS

This application claims priority from U.S. provisional patent applications 63/246,854, filed Sep. 22, 2021, and 63/403,763, filed Sep. 4, 2022, both of which are incorporated herein by reference.

### FIELD OF THE INVENTION

The present invention relates to similarity search generally and to X-ray image search in particular.

### BACKGROUND OF THE INVENTION

When radiologists encounter an ambiguous case, they typically search in public or internal databases for similar cases that would help them in the diagnostic decision-making process. Such searches are a significant burden to their workflow, and reduces time available to diagnose other cases. It is important to replace such a manual intensive search, with an automatic content-based image retrieval system.

In their paper: “Interpretability-Guided Content-Based Medical Image Retrieval” by Wilson Silva, Alexander Poellinger, Jaime S. Cardoso and Mauricio Reyes, at MICCAI 2020, Silva et al describe a medical image retrieval system 100 as shown in FIG. 1. System 100 has a convolutional neural network (CNN) disease classifier 103 and a K-Nearest Neighbor (KNN) searcher 105. CNN disease classifier 103 is a CNN that was trained using a publicly available chest X-ray image training dataset. A plurality of candidate diagnosed chest X-rays 101 from the same publicly available set were encoded into a plurality of candidate diagnosed embeddings 102, using CNN disease classifier 103, as described in the paper.

KNN searcher 105 then performed a KNN search using candidate diagnosed embeddings 102 against a query partially diagnosed X-ray 107 which had similarly been encoded into a query partially diagnosed embedding 108. As a result, K (for example 10) candidate diagnosed embeddings 102 that were most similar to the query partially diagnosed X-ray 107 were returned by KNN searcher 105. System 100 then returned the candidate diagnosed chest X-rays 101 associated with the K candidate diagnosed embeddings 102 to the operator, as the K most cases in the database, most similar to the partially diagnosed X-ray 107.

### SUMMARY OF THE PRESENT INVENTION

There is therefore provided, in accordance with a preferred embodiment of the present invention a system to retrieve medical X-rays. The system includes a trained convolutional neural network (CNN), a balancing feature generator, a balancing type selector, and a K-Nearest Neighbor (KNN) classifier. The trained CNN encodes a plurality of diagnosed X-ray images into a plurality of candidate embeddings, and encodes a partially diagnosed X-ray image into a query embedding. The balancing feature generator produces a plurality of virtual candidate embeddings from the query embedding and the plurality of candidate embeddings. The balancing type selector selects a subset of the plurality of virtual candidate embeddings. The KNN classifier performs a KNN search between the query embedding and a plurality of the candidate embeddings and the subset of the plurality of virtual candidate embeddings.

Moreover, in accordance with a preferred embodiment of the present invention, the system includes a diagnosed X-ray image datastore, an embeddings datastore, and a balancing embeddings datastore. The diagnosed X-ray image datastore stores the plurality of diagnosed X-ray images, the embeddings datastore stores the plurality of candidate embeddings, and a balancing embeddings datastore. The balancing embeddings datastore stores the plurality of virtual candidate embeddings.

Further, in accordance with a preferred embodiment of the present invention, the system includes a target diagnosis selector which filters unwanted candidate embeddings stored in the embeddings datastore, from the KNN classifier, prior to the performance of the KNN search.

Still further, in accordance with a preferred embodiment of the present invention, the system includes a data visualizer which shows the quantity of the plurality of candidate embeddings stored in the embeddings datastore, and/or the quantity of the plurality of virtual candidate embeddings stored in the balancing embeddings datastore, that are associated with a plurality of diagnoses and a plurality of classes of the plurality of diagnoses.

Additionally, in accordance with a preferred embodiment of the present invention, the system includes an X-ray data retriever which retrieves diagnostic and image data, from the diagnosed image X-ray datastore, that is associated with the K nearest neighbor candidates returned by the KNN classifier during the KNN search.

Moreover, in accordance with a preferred embodiment of the present invention, the system is implemented in associative memory.

There is also provided, in accordance with a preferred embodiment of the present invention, a method to retrieve medical X-rays. The method includes encoding a plurality of diagnosed X-ray images into a plurality of candidate embeddings, and second encoding a partially diagnosed X-ray image into a query embedding, producing a plurality of virtual candidate embeddings from the query embedding and the plurality of candidate embeddings, selecting a subset of the plurality of virtual candidate embeddings, and performing a KNN search between the query embedding and a plurality of the candidate embeddings and the subset of the plurality of virtual candidate embeddings.

Moreover, in accordance with a preferred embodiment of the present invention, the method includes storing the plurality of diagnosed X-ray images in a diagnosed X-ray image datastore, storing the plurality of candidate embeddings in an embeddings datastore, and storing the plurality of virtual candidate embeddings in a balancing embeddings datastore.

Further, in accordance with a preferred embodiment of the present invention, the method includes filtering unwanted candidate embeddings stored in the embeddings datastore, from the KNN classifier, prior to the performance of the KNN search.

Still further, in accordance with a preferred embodiment of the present invention, the method includes showing the quantity of the plurality of candidate embeddings stored in the embeddings datastore, and/or the quantity of the plurality of virtual candidate embeddings stored in the balancing embeddings datastore, that are associated with a plurality of diagnoses and a plurality of classes of the plurality of diagnoses.

Additionally, in accordance with a preferred embodiment of the present invention, the method includes retrieving diagnostic and image data, from the diagnosed image X-ray datastore, that is associated with the K nearest neighbor candidates returned by the KNN classifier during the KNN search.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a schematic illustration of a prior art X-ray image retrieval system;

FIG. 2 is a schematic illustration of a balancing X-ray image retrieval system, constructed and operative in accordance with a preferred embodiment of the present invention;

FIG. 3A is a schematic illustration of a balancing X-ray image retrieval system implemented on an associative processing unit, constructed and operative in accordance with a preferred embodiment of the present invention; and

FIG. 3B is a schematic illustration of a balancing X-ray image retrieval system implemented on an associative processing unit, constructed and operative in accordance with a preferred embodiment of the present invention.

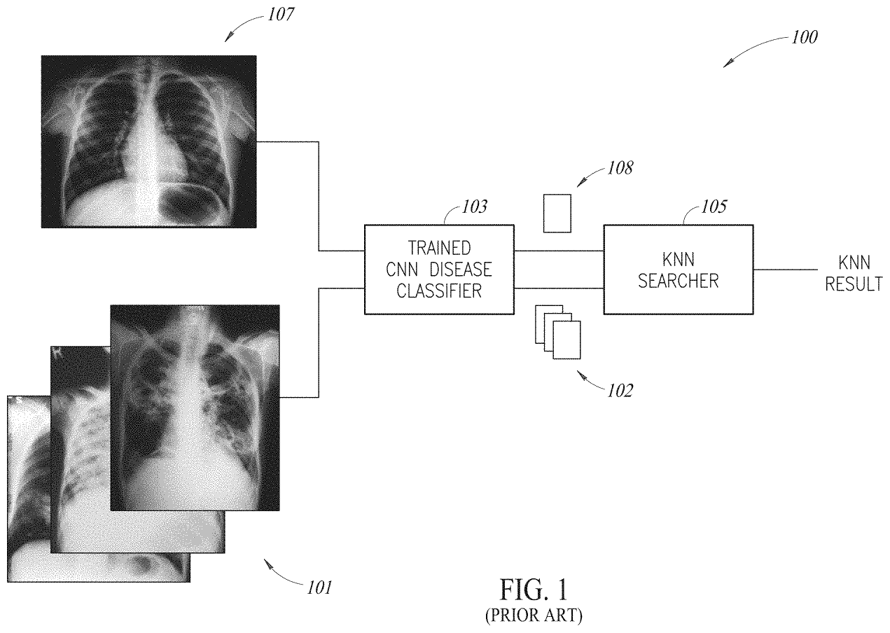

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Applicant has realized that for accurate KNN search, the candidate dataset (against which a query will be searched) needs to be balanced. To be balanced, a dataset does not have an overwhelming amount of data for only one, or only some of the target candidate classes or groups. The problem with Silva et Al's X-ray CNN/KNN system described hereinabove, is that the dataset of candidate X-ray embeddings is unbalanced. The imbalance is reflected in that for any particular diagnosis, or class of diagnosis (which may be the class or group mentioned hereinabove), there number of records associated with each class or group, is not equal. For example, if there are 5 diagnosis classes, 1 thru 5, the number of X-ray records associated with the groups is unequal.

Such an imbalance in diagnosed candidate X-ray records leads to an imbalance in candidate X-ray embeddings. This imbalance leads to deterioration of the performance of the Silva et Al's KNN X-ray diagnosis method.

The article ‘Smote-variants: a Python Implementation of 85 Minority Oversampling Techniques, in Neurocomputing Journal, June 2019, describes methods to create ‘virtual-embeddings’ from existing embeddings, so as to increase the number of available embeddings.

Applicant has realized that the methods used to create ‘virtual-embeddings’ described in the abovementioned article, may also be used to create ‘virtual candidate X-ray embeddings.’

Applicant has realized that by adding a ‘balancing system’ to an X-ray CNN/KNN system, the accuracy of prediction results may be improved.

Applicant has realized that by enabling users to choose between KNN search results both with and without additional virtual embeddings, they may choose the more accurate result.

### CNN/KNN X-ray Retrieval System

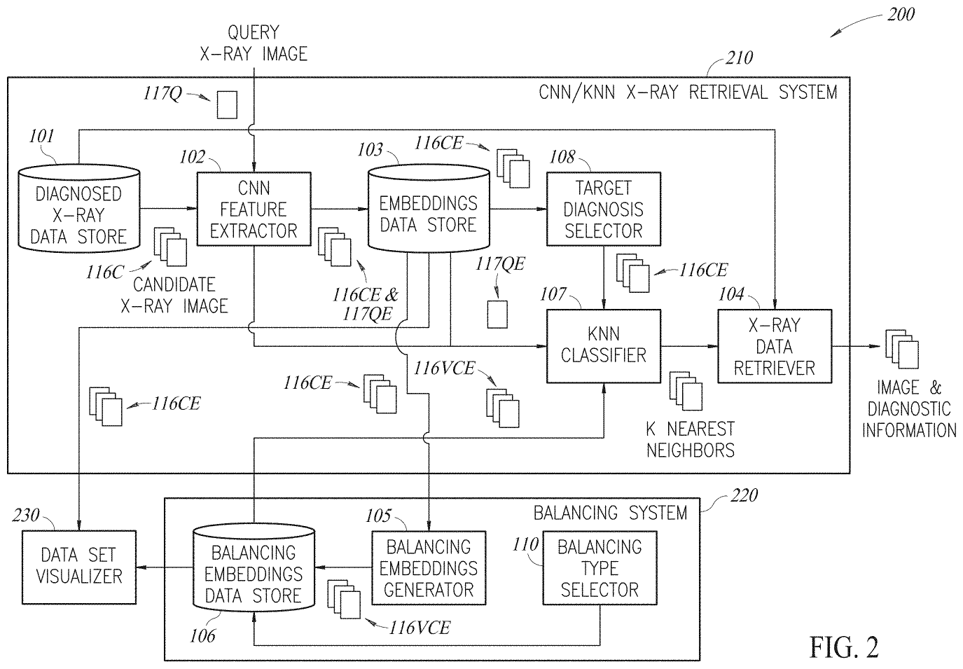

Reference is made to FIG. 2 which illustrates a balancing X-ray image retrieval system 200. System 200 comprises a CNN/KNN X-ray retrieval system 210, a balancing system 220, and a dataset visualizer 230. CNN/KNN X-ray retrieval system 210 comprises a diagnosed X-ray image datastore 101, a CNN feature extractor 102, an embeddings datastore 103, a target diagnosis selector 108, a KNN classifier 107, and an X-ray data retriever 104.

Utilizing an image KNN system like that described in U.S. Pat. No. 10,929,751, entitled “FINDING K EXTREME VALUES IN CONSTANT PROCESSING TIME” issued Feb. 23, 2021, owned by Applicant, and incorporated here by reference, a plurality of known candidate X-ray images 116C from diagnosed X-ray datastore 101, and an unknown query X-ray image 117Q may be encoded into candidate X-ray embeddings 116CE and query X-ray embedding 117QE respectively, by CNN feature extractor 102, and may be stored in a embeddings datastore 103. Candidate X-ray embeddings 116CE and query X-ray embeddings 117QE may then be input into a KNN classifier 107 for identification.

It will be appreciated that diagnosed or candidate X-ray images 116C and their associated candidate X-ray embeddings 116CE may represent different classes of diagnoses such as cancers, viral infections, bacterial infections, etc. It will also be appreciated that diagnosed X-ray images 116C and their associated candidate X-ray embeddings 116CE may also represent different diagnoses within such classes of diagnoses, for example, different cancer types.

A radiologist who may suspect, for example, a particular cancer type, may want to exclude candidate X-ray embeddings 116CE associated with non-cancer diagnoses from KNN classifier 107. She may view a visualization of the candidate X-ray embeddings 116CE dataset contained in embedding datastore 103 utilizing data visualizer 230. Such a visualization may show the number of X-ray embeddings 116CE associated with a plurality of diagnoses and a plurality of classes of diagnoses. With a knowledge of such numbers of candidate X-ray embeddings 116CE, she may then exclude any unwanted candidate X-ray embeddings 116CE using target diagnosis selector 108. Target diagnosis selector 108 may select only candidate X-ray embeddings 116CE from embeddings datastore 103 that match, for example, the suspected or target diagnosis class, and may input such candidate X-ray embeddings 116CE into KNN classifier 107. It will be appreciated that the radiologist may alternatively choose not to filter the dataset, and hence may input no data requirements into target diagnosis selector 108.

KNN classifier 107 may then find K candidate X-ray embeddings 116CE which are nearest neighbors to query X-ray embedding 117QE. X-ray data retriever 104 may then retrieve diagnostic and image data associated with the K nearest neighbor candidates from diagnosed X-ray datastore 101, and may then output the image and diagnostic information that corresponds to the K nearest neighbors returned by KNN classifier 107.

### Balancing System

Balancing system 220 comprises a balancing embeddings generator 105, a balancing embeddings datastore 106, and a balancing type selector 110.

In the abovementioned operational scenario, after reviewing a visualization of candidate X-ray embeddings 116CE on dataset visualizer 230, the radiologist may consider that the number of candidate X-ray embeddings 116CE for any particular diagnosis or class (for example, a particular lung cancer type) in embeddings datastore 103 is too low to produce an accurate KNN calculation or classification. In such a case, she may choose to add a plurality of virtual candidate X-ray embeddings 116VCE, to the plurality of candidate embeddings 116CE, used by KNN classifier 107 in the KNN calculation.

### Balancing Utilizing Existing Virtual Candidate X-ray Embeddings

To balance the candidate dataset, the radiologist may add a plurality of existing virtual candidate X-ray embeddings 116VCE from balancing embeddings datastore 106. She may enter the required number and type(s) of virtual candidate X-ray embeddings 116VCE on balancing type selector 110, which will add that number and type(s) from balancing embeddings datastore 106 to KNN classifier 107. The radiologist may them repeat the KNN classification, using the balanced data set, in a similar manner to described above.

It will be appreciated that by changing the number and type of virtual candidate X-ray embeddings 116VCE to be input to KNN classifier 107 by balancing type selector 110 between ‘no additional virtual candidate X-ray embeddings 116VCE’ and a ‘desired number of additional virtual candidate X-ray embeddings 116VCE’, the radiologist may now compare the KNN search results produced by the original unbalanced data set using only selected candidate X-ray embeddings 116CE, and the result produced by the balanced data set with additional virtual candidate X-ray embeddings 116VCE. The radiologist may then compare KNN search results both with and without additional virtual embeddings and may then choose the more accurate result.

### Generating New Virtual Candidate X-ray Embeddings

If there are not enough virtual candidate X-ray embeddings 116VCE in balancing embeddings datastore 106, the radiologist may choose to create some new virtual candidate X-ray embeddings 116VCE. She may enter into balancing embeddings generator 105, the number of virtual candidate X-ray embeddings 116VCE she wishes to create and the type of candidate X-ray embedding 116CE from which she wishes them created. Balancing embeddings generator 105 may search in feature datastore 103 for m (for example m=5) nearest neighbor candidate X-ray embeddings 116CE to query X-ray embedding 117QE. Balancing embeddings generator 105 may then generate a new virtual candidate X-ray embedding 116VCE that has feature vectors that are, for example but not limited to, an average of the m candidate X-ray embeddings 116CE, found by the algorithm.

Balancing embeddings generator 105 may store virtual candidate X-ray embedding 116VCE in balancing embeddings datastore 106. This process may be repeated as often as required. It will be appreciated that due to the random nature of KNN search, the generation of a plurality of virtual candidate X-ray embeddings 116VCE, from the same KNN search against the same query X-ray embedding 117QE by balancing embeddings generator 105, may not produce identical virtual candidate X-ray embeddings 116VCE.

### Associative Processor Balancing X-ray Image Retrieval System

Balancing X-ray image system 200 may be implemented on an associative memory array within an associative processing unit, similar to the KNN system in U.S. Pat. No. 10,929,751 mentioned hereinabove. The massive parallel processing functionality of associative processing units may reduce data manipulation and KNN search times.

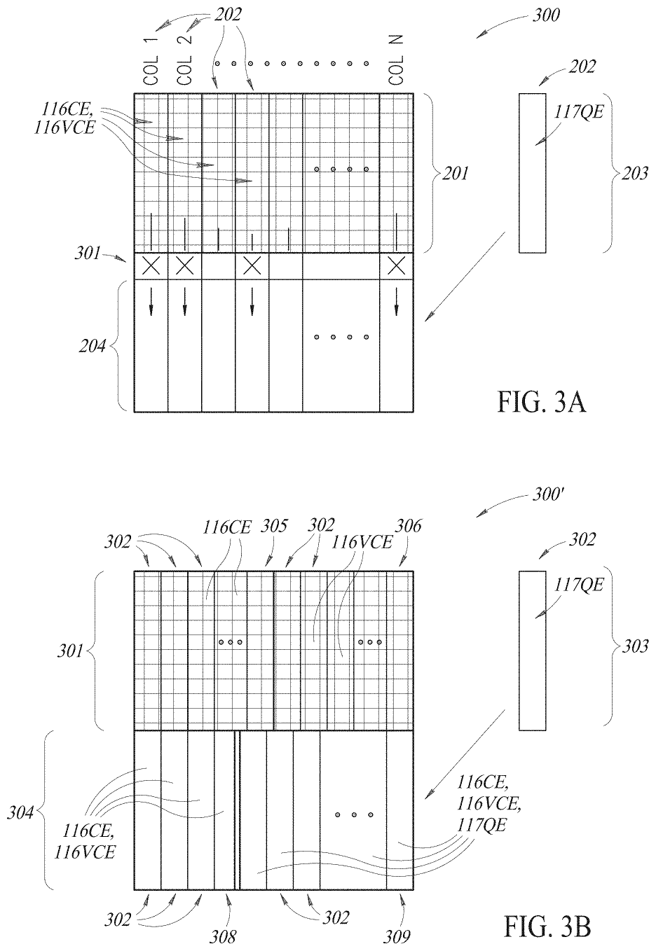

Reference is made to FIG. 3A which illustrates a preferred embodiment of the present invention implemented on an associative processing unit (APU) 300. APU 300 may be any suitable APU such as the Gemini APU, commercially available from GSI Technology Inc. of the USA. APU 300 may comprise a datastore 201 (which has been shaded for clarity) in a portion of APU 300, a KNN classifier 204 in another portion of APU 300, a query store 203 in a third portion of APU 300, and a marker row 301. It should be noted that datastore 201, KNN classifier 204, query store 203, and marker row 301 may be in any part of APU 300, and may even be mixed together. Datastore 201 and query store 203 may comprise a plurality of columns 202. A plurality of candidate X-ray embeddings 116CE, and a plurality of virtual candidate X-ray embeddings 116VCE may be stored in columns 202 of datastore 201. A query X-ray embedding 117QE may be stored in column 202 of query store 203.

KNN classifier 204 may operate on plurality of candidate X-ray embeddings 116CE, plurality of virtual candidate X-ray embeddings 116VCE, and query X-ray embedding 117QE in a massively parallel operation as described in U.S. Pat. No. 10,929,751, mentioned hereinabove. It will be appreciated that candidate embeddings 112 and virtual candidate embeddings 113 may be included or excluded as required by KNN classifier 204, by use of a marker row 301. When columns in marker row 301 are selected, then only those embeddings in those rows may be included in the KNN classification. Marker row 310 may be the implementation of target diagnosis selector 108 and balancing type selector 110, both of which are explained hereinabove.

Reference is made to FIG. 3B which illustrates another preferred embodiment of the present invention implemented on an APU 300′. Datastore 301 may comprise a separate candidate X-ray embedding datastore 305, and a separate balancing embedding datastore 306. KNN classifier 304 may comprise a temporary store 308 and a KNN processor 309. Candidate embedding datastore 305, balancing feature datastore 306, temporary store 308, and KNN processor 309 may comprise a plurality of columns 302. A plurality of candidate X-ray embeddings 116CE may be stored in columns 202 of candidate embedding datastore 305. A plurality of virtual candidate X-ray embeddings 116VCE may be stored in columns 202 of balancing feature datastore 306. A query X-ray embedding 117QE may be stored in column 302 of query store 303.

A query X-ray embedding 117QE, a selected plurality of candidate X-ray embeddings 116CE, and selected plurality of virtual candidate X-ray embeddings 116VCE, may be written to columns 302 of temporary store 308 before being operated on in parallel by KNN classifier 309.

It will be appreciated that through balancing datasets, the accuracy of X-ray image identification in the medical image system described by Silva et al hereinabove improved by 5% from unbalanced results.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A system to retrieve medical X-rays, the system comprising:
a trained convolutional neural network (CNN) to encode a plurality of diagnosed X-ray images into a plurality of candidate embeddings, and to encode a partially diagnosed X-ray image into a query embedding;
a balancing feature generator to produce a plurality of virtual candidate embeddings from said query embedding and said plurality of candidate embeddings;
a balancing type selector to select a subset of said plurality of virtual candidate embeddings; and
a K-Nearest Neighbor (KNN) classifier to perform a KNN search between said query embedding and a plurality of said candidate embeddings and said subset of said plurality of virtual candidate embeddings.

2. The system according to claim 1 and also comprising:
a diagnosed X-ray image datastore to store said plurality of diagnosed X-ray images;
an embeddings datastore to store said plurality of candidate embeddings; and
a balancing embeddings datastore to store said plurality of virtual candidate embeddings.

3. The system according to claim 1 and also comprising:
a target diagnosis selector to filter unwanted candidate embeddings stored in said embeddings datastore, from said KNN classifier, prior to the performance of said KNN search.

4. The system according to claim 1 and also comprising:
a data visualizer to show the quantity of said plurality of candidate embeddings stored in said embeddings datastore, and/or the quantity of said plurality of virtual candidate embeddings stored in said balancing embeddings datastore, that are associated with a plurality of diagnoses and a plurality of classes of said plurality of diagnoses.

5. The system according to claim 1 and also comprising:
an X-ray data retriever to retrieve diagnostic and image data, from said diagnosed image X-ray datastore, that is associated with the K nearest neighbor candidates returned by said KNN classifier during said KNN search.

6. The system according to claim 1 implemented in associative memory.

7. A method to retrieve medical X-rays, the method comprising:
encoding a plurality of diagnosed X-ray images into a plurality of candidate embeddings, and second encoding a partially diagnosed X-ray image into a query embedding;
producing a plurality of virtual candidate embeddings from said query embedding and said plurality of candidate embeddings;
selecting a subset of said plurality of virtual candidate embeddings; and
performing a KNN search between said query embedding and a plurality of said candidate embeddings and said subset of said plurality of virtual candidate embeddings.

8. The method of claim 1 and also comprising:
storing said plurality of diagnosed X-ray images in a diagnosed X-ray image datastore;
storing said plurality of candidate embeddings in an embeddings datastore; and
storing said plurality of virtual candidate embeddings in a balancing embeddings datastore.

9. The method of claim 1 and also comprising:
filtering unwanted candidate embeddings stored in said embeddings datastore, from said KNN classifier, prior to the performance of said KNN search.

10. The method of claim 1 and also comprising:
showing the quantity of said plurality of candidate embeddings stored in said embeddings datastore, and/or the quantity of said plurality of virtual candidate embeddings stored in said balancing embeddings datastore, that are associated with a plurality of diagnoses and a plurality of classes of said plurality of diagnoses.

11. The method of claim 1 and also comprising:
retrieving diagnostic and image data, from said diagnosed image X-ray datastore, that is associated with the K nearest neighbor candidates returned by said KNN classifier during said KNN search.
